(19) United States
(12) Patent Application Publication
(10) Pub. No.: US 2025/0089988 A1
(43) Pub. Date: Mar. 20, 2025

(54) IMAGE PROCESSING APPARATUS, IMAGE PROCESSING METHOD, AND STORAGE MEDIUM

(71) Applicant: OLYMPUS MEDICAL SYSTEMS CORP., Tokyo (JP)

(72) Inventors: Kazuhito HORIUCHI, Tokyo (JP); Ryuhi OKUBO, Tokyo (JP); Kazumi ITO, Tokyo (JP); Satoru ITO, Tokyo (JP)

(73) Assignee: OLYMPUS MEDICAL SYSTEMS CORP., Tokyo (JP)

(21) Appl. No.: 18/882,543

(22) Filed: Sep. 11, 2024

Related U.S. Application Data
(60) Provisional application No. 63/538,557, filed on Sep. 15, 2023.

(51) Int. Cl. A61B 1/00; G06T 3/40; G06T 7/571; G06T 7/60; G06T 11/20

(52) U.S. Cl. CPC A61B 1/000094; A61B 1/0005; G06T 3/40; G06T 7/571; G06T 7/60; G06T 11/203; G06T 11/206; G06T 2207/10068; G06T 2210/41

(57) ABSTRACT

An image processing apparatus for use with an endoscope, and including a processor comprising hardware. The processor is configured to obtain an object distance, calculate dimensional characteristics defining apparent dimensions of an object in an image acquired by the endoscope, calculate scale information defining a relationship between the apparent dimensions and actual dimensions based on the object distance and the dimensional characteristics, and generate an indication pattern representing the actual dimensions based on the scale information.

### CROSS-REFERENCE TO RELATED APPLICATIONS

This application claims the benefit of U.S. Provisional Application No. 63/538,557, filed on Sep. 15, 2023, which is hereby incorporated by reference herein in its entirety.

### TECHNICAL FIELD

The present disclosure relates to an image processing apparatus, an image processing method, and a storage medium.

### BACKGROUND ART

A known, conventional endoscope system in the related art has a function for measuring the dimensions of an object (for example, see PTL 1). The endoscope system described in PTL 1 forms a spot on an object by irradiating the object with measurement assist light emitted from an endoscope, estimates an object distance based on the position of the spot on an imaging device, generates an index figure representing the actual dimensions of the object on the basis of the object distance, and displays the index figure together with an image of the object.

This endoscope system enables estimation of the accurate dimensions of the object without relying on the subjective judgment or skill of a doctor. In addition, the doctor is enabled to select a treatment technique depending on the dimensions of the object, such as a diseased part, in endoscopic examination and treatment.

### CITATION LIST

### Patent Literature

PTL 1: Publication of Japanese Patent No. 6692440

### SUMMARY

An aspect of the disclosure is an image processing apparatus for use with an endoscope, the image processing apparatus including a processor comprising hardware, the processor is configured to: obtain an object distance; calculate dimensional characteristics defining apparent dimensions of an object in an image acquired by the endoscope; calculate scale information defining a relationship between the apparent dimensions and actual dimensions of the object based on the object distance and the dimensional characteristics; and generate an indication pattern representing the actual dimensions based on the scale information.

Another aspect of the disclosure is an image processing apparatus for use with an endoscope, the image processing apparatus including a processor comprising hardware, wherein the processor is configured to: calculate an object distance based on a focus position of a focus lens, the focus position being a position when an object is focused; calculate dimensional characteristics defining apparent dimensions of the object based on optical system information of an imaging optical system of the endoscope in a state in which the focus lens is located at the focus position; generate three-dimensional information of the object using an image acquired by the endoscope; calculate scale information defining a relationship between the apparent dimensions and actual dimensions based on the object distance, the dimensional characteristics, and the three-dimensional information; and generate an indication pattern representing the actual dimensions based on the scale information, the indication pattern is configured to be superimposed on the image.

Another aspect of the disclosure is an image processing method for use with an endoscope, the image processing method including: calculating an object distance based on a focus position of a focus lens, the focus position being a position when an object is focused; calculating dimensional characteristics defining apparent dimensions of the object based on optical system information of an imaging optical system of the endoscope in a state in which the focus lens is located at the focus position; calculating scale information defining a relationship between the apparent dimensions and actual dimensions of the object based on the object distance and the dimensional characteristics; and generating an indication pattern representing the actual dimensions based on the scale information, the indication pattern is configured to be superimposed on an image acquired by the endoscope.

Another aspect of the disclosure is an image processing method for use with an endoscope, the image processing method including: calculating an object distance based on a focus position of a focus lens, the focus position being a position when an object is focused; calculating dimensional characteristics defining apparent dimensions of the object based on optical system information of an imaging optical system of the endoscope in a state in which the focus lens is located at the focus position; generating three-dimensional information of the object using an image acquired by the endoscope; calculating scale information defining a relationship between the apparent dimensions and actual dimensions of the object based on the object distance, the dimensional characteristics, and the three-dimensional information; and generating an indication pattern representing the actual dimensions based on the scale information, the indication pattern is configured to be superimposed on the image.

Another aspect of the disclosure is a non-transitory computer-readable storage medium storing instructions that cause a computer to perform: calculating an object distance based on a focus position of a focus lens, the focus position being a position when an object is focused; calculating dimensional characteristics defining apparent dimensions of the object based on optical system information of an imaging optical system of an endoscope in a state in which the focus lens is located at the focus position; calculating scale information defining a relationship between the apparent dimensions and actual dimensions of the object based on the object distance and the dimensional characteristics; and generating an indication pattern representing the actual dimensions based on the scale information, the indication pattern is configured to be superimposed on an image acquired by the endoscope.

Another aspect of the disclosure is a non-transitory computer-readable storage medium storing instructions that cause a computer to perform: calculating an object distance based on a focus position of a focus lens, the focus position being a position when an object is focused; calculating dimensional characteristics defining apparent dimensions of the object based on optical system information of an imaging optical system of an endoscope in a state in which the focus lens is located at the focus position; generating three-dimensional information of the object using an image acquired by the endoscope; calculating scale information defining a relationship between the apparent dimensions and actual dimensions of the object based on the object distance, the dimensional characteristics, and the three-dimensional information; and generating an indication pattern representing the actual dimensions based on the scale information, the indication pattern is configured to be superimposed on the image.

### BRIEF DESCRIPTION OF DRAWINGS

FIG. 1 shows an overall structure of an image processing apparatus and an endoscope system according to a first embodiment.

FIG. 2 is a functional block diagram of a processor of the image processing apparatus.

FIG. 3A shows an example scale indication superimposed on an image.

FIG. 3B shows another example scale indication superimposed on an image.

FIG. 3C shows another example scale indication superimposed on an image.

FIG. 4 is a flowchart of an image processing method performed by the image processing apparatus.

FIG. 5A shows an example scale indication in which the size of the indication pattern is variable, in the case where the object distance is long.

FIG. 5B shows an example scale indication in which the size of the indication pattern is variable, in the case where the object distance is short.

FIG. 6A shows another example scale indication in which the size of the indication pattern is variable, in the case where the object distance is long.

FIG. 6B shows another example scale indication in which the size of the indication pattern is variable, in the case where the object distance is short.

FIG. 7A shows an example scale indication in which the size of the indication pattern is fixed and numerical values are variable, in the case where the object distance is long.

FIG. 7B shows an example scale indication in which the size of the indication pattern is fixed and numerical values are variable, in the case where the object distance is short.

FIG. 8A shows another example scale indication in which the size of the indication pattern is fixed and numerical values are variable, in the case where the object distance is long.

FIG. 8B shows another example scale indication in which the size of the indication pattern is fixed and numerical values are variable, in the case where the object distance is short.

FIG. 9A is a diagram for explaining multiple ranging areas in an image.

FIG. 9B shows an image having distortion, on which scale indications are superimposed at the center and the periphery.

FIG. 10A shows an example image in which a scale indication is superimposed on an object on the near side.

FIG. 10B shows an example image in which a scale indication is superimposed on an object on the far side.

FIG. 11A shows another example image in which a scale indication is superimposed on an object on the near side.

FIG. 11B shows another example image in which a scale indication is superimposed on an object on the far side.

FIG. 12A shows another example image in which a scale indication is superimposed on an object on the near side.

FIG. 12B shows another example image in which a scale indication is superimposed on an object on the far side.

FIG. 13A shows another example image in which a scale indication is superimposed on an object on the near side.

FIG. 13B shows another example image in which a scale indication is superimposed on an object on the far side.

FIG. 14A shows another example image in which a scale indication is superimposed on an object on the near side.

FIG. 14B shows another example image in which a scale indication is superimposed on an object on the far side.

FIG. 15 is a functional block diagram of a processor of an image processing apparatus according to a second embodiment.

FIG. 16 is a flowchart of an example method for generating 3D information.

FIG. 17A shows an example image in which an object on the near side is focused, and a scale indication is superimposed at the center.

FIG. 17B shows an example image in which an object on the far side is focused, and a scale indication is superimposed at the center.

FIG. 18A shows an example image on which a scale indication is superimposed, in the case where a measurement point is set on an object on the near side.

FIG. 18B shows an example image on which a scale indication is superimposed, in the case where a measurement point is set on an object on the far side.

FIG. 19 is a flowchart of an image processing method according to a second embodiment.

### DESCRIPTION OF EMBODIMENTS

### First Embodiment

A conventional endoscope system requires measurement assist light in measuring the dimensions of the object. Hence, it is necessary to use a special endoscope equipped with an illumination optical system for the measurement assist light. Such an endoscope is not suited to reducing its diameter.

An image processing apparatus, an image processing method, and a storage medium according to a first embodiment of the present disclosure will be described with reference to the drawings.

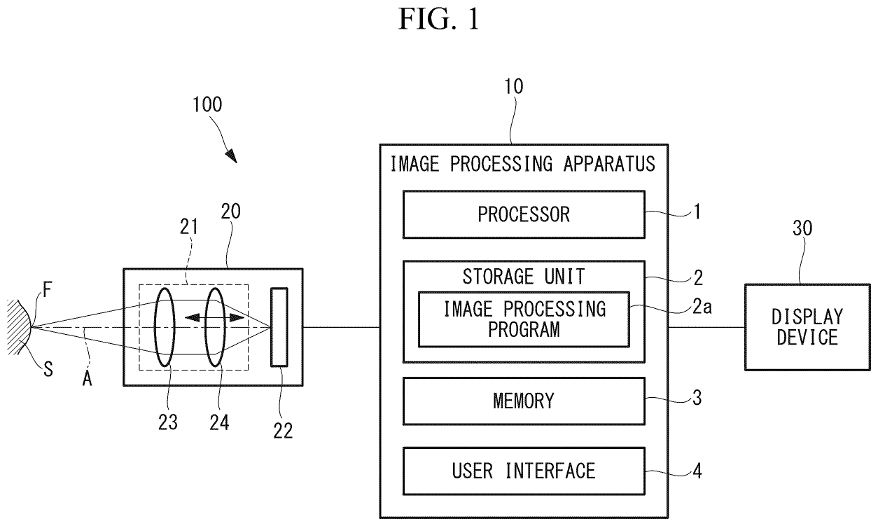

As shown in FIG. 1, an image processing apparatus 10 according to this embodiment is applied to an endoscope system 100.

The endoscope system 100 includes the image processing apparatus 10, an endoscope 20, and a display device 30. The image processing apparatus 10 is connected to the endoscope 20 and the display device 30.

The endoscope 20 includes an imaging optical system 21 and an imager 22. The imaging optical system 21 includes an objective lens 23 at the distal end of the endoscope 20, and an autofocus (AF) lens 24 between the objective lens 23 and the imager 22.

The imager 22, which includes an image sensor such as a CMOS image sensor, captures an image of an object S through the imaging optical system 21 and generates an image. The image output from the imager 22 is input to the display device 30 through the image processing apparatus 10 and is displayed on the display device 30. The display device 30 is any type of display device such as a liquid crystal display.

The AF lens 24 is movable along an optical axis A of the objective lens 23. A focal point F of the objective lens 23 is moved on the optical axis A by the movement of the AF lens 24. The endoscope 20 can perform an AF operation of moving the AF lens 24 along the optical axis A with an actuator (not shown) to focus on the object S. The endoscope 20 may always automatically perform the AF operation, or may perform the AF operation in response to a control signal from the outside. The endoscope 20 may perform the AF operation so as to focus on a position in the image designated by the user.

The image processing apparatus 10 includes a processor 1 such as a central processing unit, a storage unit 2, a memory 3, and a user interface 4.

The storage unit 2 is a non-transitory computer-readable storage medium, and examples thereof include a known magnetic disk, optical disk, and flash memory. The storage unit 2 stores an image processing program 2a for causing the processor 1 to perform the image processing method described below.

The memory 3 is a volatile storage device, such as a random access memory (RAN), and is used as a work area for the processor 1.

The user interface 4 includes input devices such as a mouse, a keyboard, a touch panel, and a microphone. A user can input various information, instructions, and the like to the image processing apparatus 10 by operating the user interface 4.

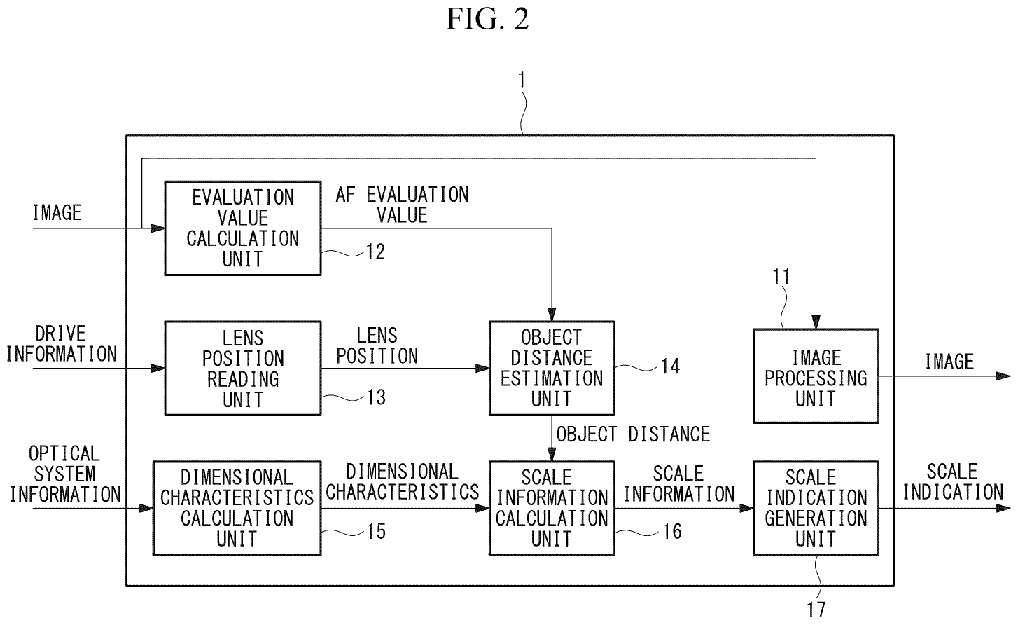

FIG. 2 is a functional block diagram of the processor 1. The processor 1 includes, as functions, an image processing unit 11, an evaluation value calculation unit 12, a lens position reading unit 13, an object distance estimation unit 14, a dimensional characteristics calculation unit 15, a scale information calculation unit 16, and a scale indication generation unit 17.

The image processing unit 11 acquires an image input from the endoscope 20 to the image processing apparatus 10, performs image processing including missing pixel correction, white balance adjustment, noise reduction, and demosaicing on the image, and then outputs the image to the display device 30.

The evaluation value calculation unit 12, the lens position reading unit 13, the object distance estimation unit 14, the dimensional characteristics calculation unit 15, the scale information calculation unit 16, and the scale indication generation unit 17 perform following processing while a measurement mode for measuring the dimensions of the object S is performed. The processor 1 performs the measurement mode in response to, for example, a start instruction. The start instruction is input to the image processing apparatus 10 when, for example, the user operates the user interface 4 at a desired timing.

The evaluation value calculation unit 12 successively acquires images during the AF operation and the images are input from the endoscope 20 to the image processing apparatus 10, and successively calculates AF evaluation values, which indicate the degree of focus of each of the images, using the images. For example, the AF evaluation value is the contrast of an image. The contrast peaks when the object S is focused.

The evaluation value calculation unit 12 may calculate the AF evaluation values using phase difference information instead of the images. In that case, the imager 22 has pixels used to detect an image plane phase difference, and the phase difference information is input from the endoscope 20 to the image processing apparatus 10.

The lens position reading unit 13 acquires drive information of the AF lens 24, including the position of the AF lens 24 during the AF operation, from the endoscope 20, and reads the position of the AF lens 24 from the drive information.

The object distance estimation unit 14 determines the focus position of the AF lens 24 on the basis of the AF evaluation value and estimates the object distance on the basis of the focus position. The focus position is the position of the AF lens 24 in a focused state, in which the focal point F is on the object S. The object distance is the actual distance between the objective lens 23 and the object S in the direction along the optical axis A.

More specifically, the object distance estimation unit 14 determines the AF evaluation value at the time when focus is achieved, from among the AF evaluation values calculated during the AF operation. For example, when the AF evaluation value is the contrast of the image, the object distance estimation unit 14 determines the peak contrast. Next, the object distance estimation unit 14 sets, as the focus position, the position of the AF lens 24 at the time of acquisition of the image with which the AF evaluation value at the time when focus is achieved is calculated. Next, the object distance estimation unit 14 estimates the object distance from the focus position by using a lookup table (LUT). The LUT represents the relationship between the focus position and the object distance and is stored in the storage unit 2 in advance.

The dimensional characteristics calculation unit 15 acquires optical system information of the imaging optical system 21 from the endoscope 20 and calculates the dimensional characteristics of the object S in the image on the basis of the optical system information.

The optical system information is information about the optical characteristics of the imaging optical system 21 and includes the observation magnification, the amount of distortion, and the position of the optical axis center. Because the optical system information varies in accordance with the position of the AF lens 24, the dimensional characteristics calculation unit 15 acquires the optical system information in a state in which the AF lens 24 is located at each of the various positions. The dimensional characteristics calculation unit 15 may automatically acquire the optical system information before the measurement mode is performed, for example, when the endoscope 20 is connected to the image processing apparatus 10.

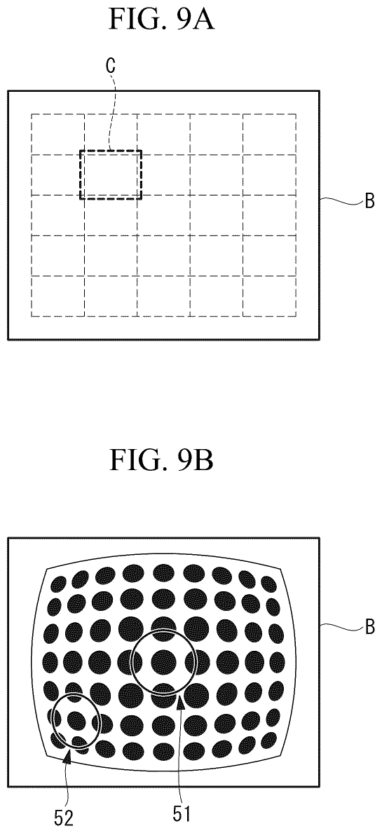

Next, the dimensional characteristics calculation unit 15 calculates the dimensional characteristics on the basis of the optical system information in the focused state, in which the AF lens 24 is located at the focus position. For example, in the case of a wide angle objective lens 23, the apparent dimensions of the object S in the image vary depending on the position due to peripheral distortion generated in the image, deviation of the position of the optical axis center, and the like. For example, the apparent dimensions decrease from the center toward the periphery (see FIG. 9B). The dimensional characteristics define these apparent dimensions of the object S at the respective positions in the image.

The scale information calculation unit 16 calculates scale information on the basis of the object distance and the dimensional characteristics. The scale information is information defining the relationship between the apparent dimensions and the actual dimensions of the object S in the image, and is, for example, information representing the relationship between the apparent distance from the optical axis center and the actual distance from the optical center in the image. The scale information calculation unit 16 calculates the actual dimensions of the focused object S on the basis of the object distance, and calculates the scale information on the basis of the actual dimensions and the dimensional characteristics of the object S.

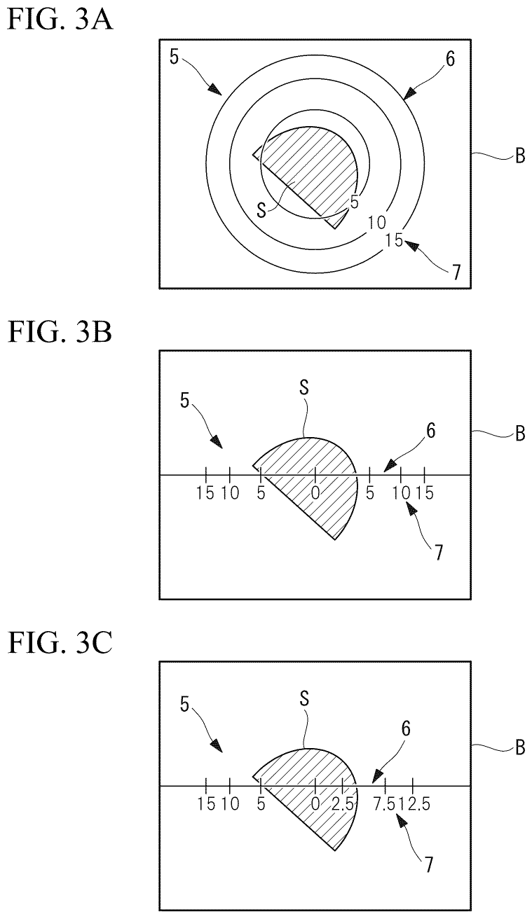

The scale indication generation unit 17 generates the scale indication 5, which is a virtual scale superimposed on the image, on the basis of the scale information. FIGS. 3A and 3B show examples of the scale indication 5. The scale indication 5 includes an indication pattern 6, which is a graphic representing the actual dimensions of the object S, and numerical values 7 representing the actual dimensions and attached to the indication pattern 6. The processor 1 outputs the image B to the display device 30 together with the scale indication 5, and causes the display device 30 to display the image B on which the scale indication 5 is superimposed.

The indication pattern 6 in FIG. 3A is concentric circles including multiple circles. The circles have mutually different radii and have centers located at the center of the image B. The radius of each circle represents the actual dimensions of the object S, and each circle is assigned a numerical value 7 representing the actual dimensions (5 mm, 10 mm, 15 mm).

The indication pattern 6 in FIG. 3B is a linear scale and has an origin located at the center of the image B. The distance from the origin represents the actual dimensions of the object S, and each scale is assigned a numerical value 7 representing the actual dimensions (5 mm, 10 mm, 15 mm). In FIG. 3B, the scales are arranged symmetrically with respect to the origin.

As shown in FIG. 3C, the scales may be arranged asymmetrically with respect to the origin. This makes it possible to measure the size of the object S more accurately.

Next, an image processing method performed by the image processing apparatus 10 will be described.

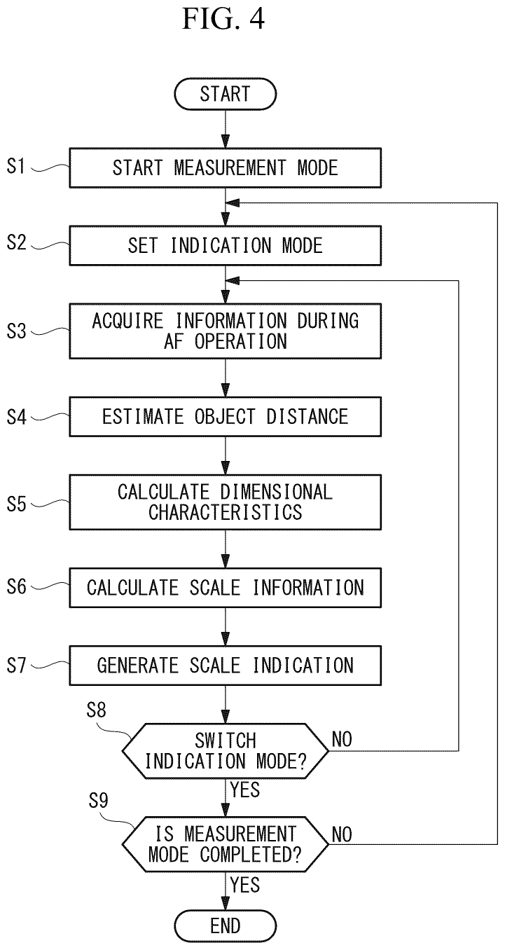

As shown in FIG. 4, the image processing method according to this embodiment includes step S3 of acquiring necessary information during the AF operation of the endoscope 20, step S4 of estimating the object distance, step S5 of calculating the dimensional characteristics of the object S in the image, step S6 of calculating the scale information, and step S7 of generating the scale indication 5.

The processor 1 starts the measurement mode in response to, for example, a start instruction (step S1), and performs steps S3 to S7.

Before step S3, the processor 1 may set an indication mode on the basis of the operation of the user interface 4 by a user (step S2). The settings of the indication mode include the type of the scale indication 5. For example, the user can select the type from the concentric circles and the linear scale.

Next, the processor 1 acquires information necessary for generating the scale indication 5, namely, an image, drive information, and optical system information, from the endoscope 20 during the AF operation of the endoscope 20 (step S3). In step S3, the processor 1 may cause the endoscope 20 to perform the AF operation by transmitting a control signal to the endoscope 20. Alternatively, the processor 1 may acquire the aforementioned information while the endoscope 20 is automatically performing the AF operation.

Next, the processor 1 estimates the object distance on the basis of the image and the drive information (step S4). More specifically, the evaluation value calculation unit 12 calculates the AF evaluation value on the basis of the image, and at the same time, the lens position reading unit 13 reads the position of the AF lens 24 from the drive information. Subsequently, the object distance estimation unit 14 determines the focus position of the AF lens 24 on the basis of the AF evaluation value, and estimates the object distance on the basis of the focus position.

Next, the dimensional characteristics calculation unit 15 calculates the dimensional characteristics of the object S in the image on the basis of the optical system information of the imaging optical system 21 in the focused state (step S5).

Next, the scale information calculation unit 16 calculates scale information on the basis of the dimensional characteristics and the object distance (step S6).

Next, the scale indication generation unit 17 generates the scale indication 5 corresponding to the set indication mode on the basis of the scale information (step S7).

The scale indication 5 is output from the image processing apparatus 10 to the display device 30 together with the image B. The display device 30 displays the image B on which the scale indication 5 is superimposed. A user, such as a doctor, can measure the actual dimensions of the object S, such as a diseased part, on the basis of the scale indication 5 on the image.

After step S7, the processor 1 may determine whether to switch the indication mode (step S8) and whether to end the measurement mode (step S9).

For example, when a user wishes to change the indication mode, the user selects a desired indication mode by operating the user interface 4. The processor 1 switches the indication mode (YES in step S8, NO in step S9, and step S2) on the basis of the operation of the user interface 4, and repeats steps S3 to S7. When the indication mode is not switched (NO in step S8), the processor 1 returns to step S3.

When the user wishes to end the measurement mode, the user inputs an end instruction to the image processing apparatus 10 by operating the user interface 4. The processor 1 ends the measurement mode in response to the end instruction (YES in step S9).

To calculate the scale information, the object distance, which is information of the actual dimensions of the object S in the image, is required. According to this embodiment, the object distance is calculated from the focus position of the AF lens 24. Hence, the image processing apparatus 10 can be combined with a typical small-diameter endoscope 20 having a focus lens such as the AF lens 24, and can measure the actual dimensions of the object S without requiring a special endoscope such as an endoscope including an illumination optical system for measurement assist light.

In this embodiment, the scale indication 5 may be variable according to the object distance.

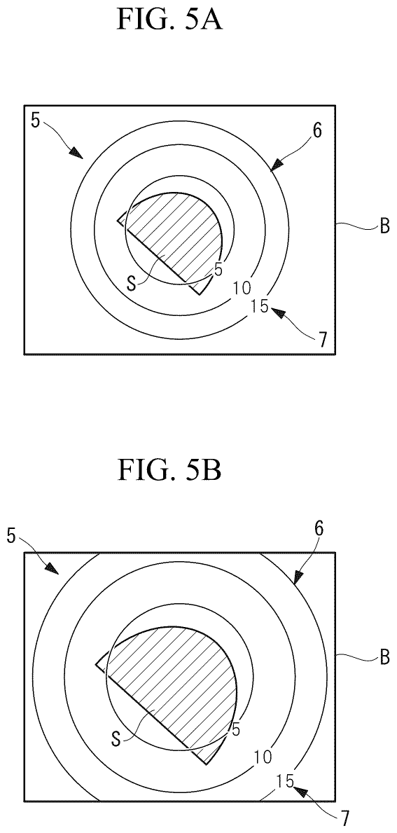

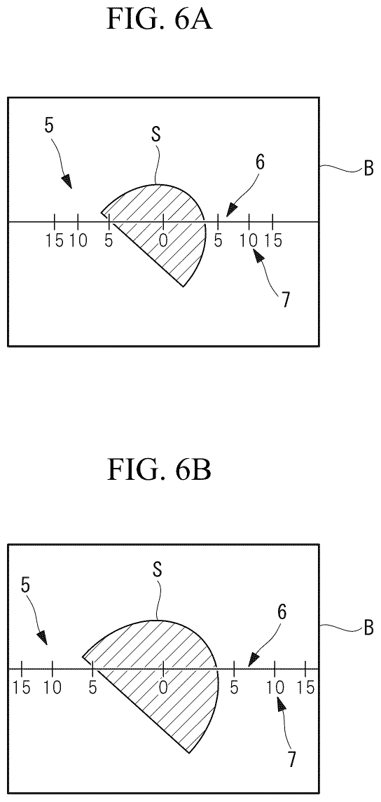

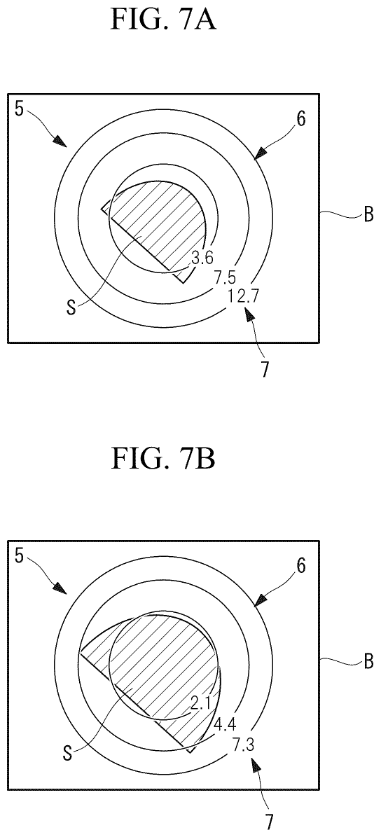

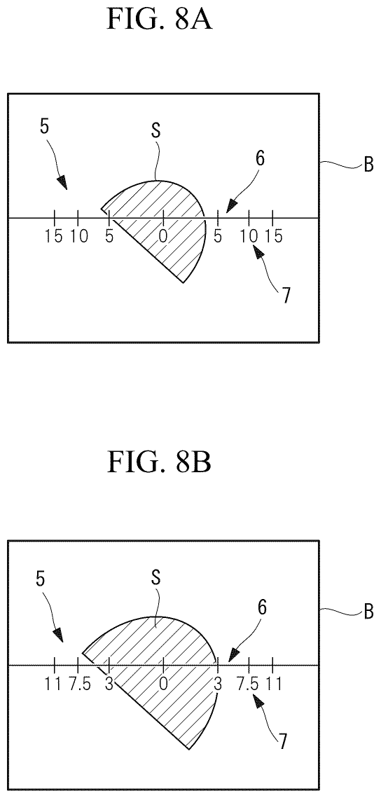

More specifically, the processor 1 may change the size of the indication pattern 6 according to the object distance (see FIGS. 5A to 6B), or may generate the indication pattern 6 having a fixed size and change the numerical values 7 according to the object distance (see FIGS. 7A to 8B).

In this case, in setting the indication mode in step S2, a user may select whether the size of the indication pattern 6 is variable or fixed.

In FIGS. 5A and 5B, the radius of each circle of the concentric circles changes with the estimated object distance. More specifically, the radius increases as the object distance decreases, by which a user recognizes that the distance to the object S is short.

In FIGS. 6A and 6B, the distance between the scales changes with the estimated object distance. More specifically, the distance between the scales increases as the object distance decreases, by which a user recognizes that the distance to the object S is short.

In FIGS. 7A to 8B, the numerical values 7 decrease as the object distance decreases, by which a user recognizes that the distance to the object S is short. The radius of each circle or the distance between the scales is constant regardless of the object distance.

In this embodiment, it has been described that one scale indication 5 is disposed at the center of the image B. Instead of this, one or more scale indications 5 may be disposed at other positions of the image B.

For example, as shown in FIGS. 9A and 9B, multiple ranging areas C arranged in the horizontal direction and the vertical direction are set in the image B, and scale indications 51 and 52 are shown at one or more focused ranging areas C. FIG. 9B shows an image B of multiple circles having the same diameter and arranged at equal intervals in the horizontal direction and the vertical direction. The circles in the periphery contract and are deformed into ellipses by distortion.

When the image B has distortion as in FIG. 9B, the scale indication generation unit 17 may generate the scale indications 51 and 52 by taking the distortion into account. In the example in FIG. 9B, each of the scale indications 51 and 52 is a single circle or ellipse, and the radius or the major and minor radii represent the actual dimensions of the object S. The scale indication 51 at the center, where no distortion occurs, is a circle. The scale indication 52 at the lower left portion, where distortion occurs, is an ellipse inclined in the direction of distortion due to a circle being distorted according to the distortion. The two scale indications 51 and 52 represent the same actual dimensions.

In this case, the object distance estimation unit 14 selects at least one focused ranging area C, and estimates the object distance of the selected ranging area C. The scale indication generation unit 17 generates the scale indications (51, 52, etc.) depending on the selected ranging area C.

The object distance estimation unit 14 may select a focused ranging area C on the basis of the AF evaluation value for each ranging area C. In that case, the evaluation value calculation unit 12 calculates the AF evaluation value for each ranging area C.

Alternatively, the object distance estimation unit 14 may select, as the focused ranging area C, the ranging area including the position designated by a user. In that case, the processor 1 causes the endoscope 20 to perform the AF operation such that the selected ranging area C is focused.

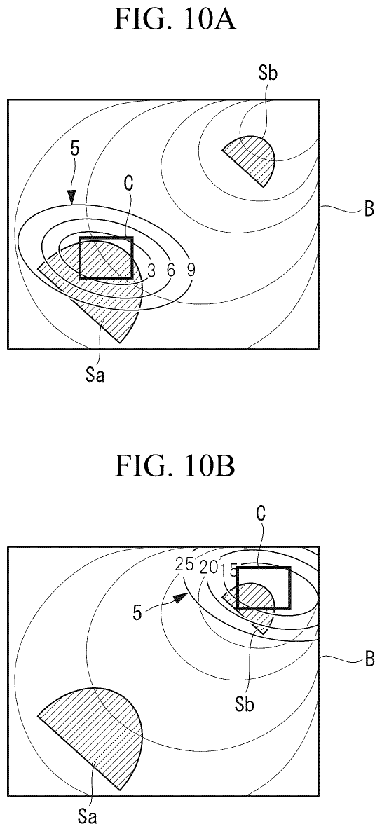

FIGS. 10A to 13B show an image B of the inside of a lumen, such as a colon, extending from the lower left to the upper right. Multiple objects Sa and Sb, such as tumors, are present at different positions in the depth direction.

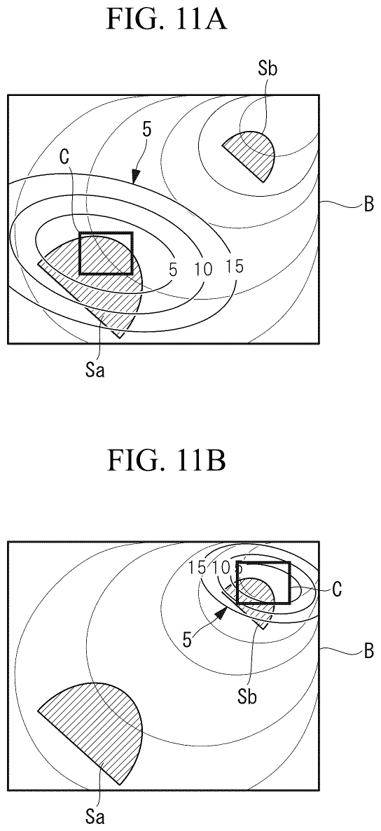

FIGS. 10A to 11B show cases in which the scale indication 5 is concentric circles. In FIGS. 10A and 10B, the size of the indication pattern 6 is fixed, whereas in FIGS. 11A and 11B, the size of the indication pattern 6 is variable. In FIGS. 10A to 11B, the indication pattern 6 includes inclined concentric ellipses resulting from concentric circles being distorted according to the distortion of the image B.

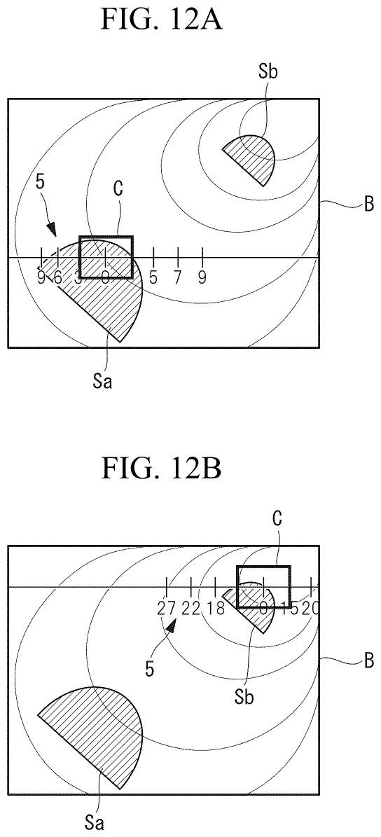

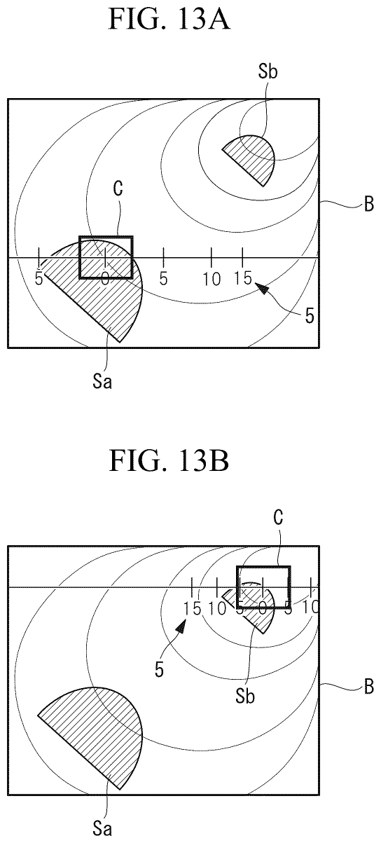

FIGS. 12A to 13B show cases in which the scale indication 5 is a linear scale. In FIGS. 12A and 12B, the size of the indication pattern 6 is fixed, whereas in FIGS. 13A and 13B, the size of the indication pattern 6 is variable.

In FIGS. 10A, 11A, 12A, and 13A, the object Sa on the near side is focused automatically or in accordance with designation by a user, and the scale indication 5 is superimposed on the object Sa. In FIGS. 10B, 11B, 12B, and 13B, the object Sb on the far side is focused automatically or in accordance with designation by a user, and the scale indication 5 is superimposed on the object Sb.

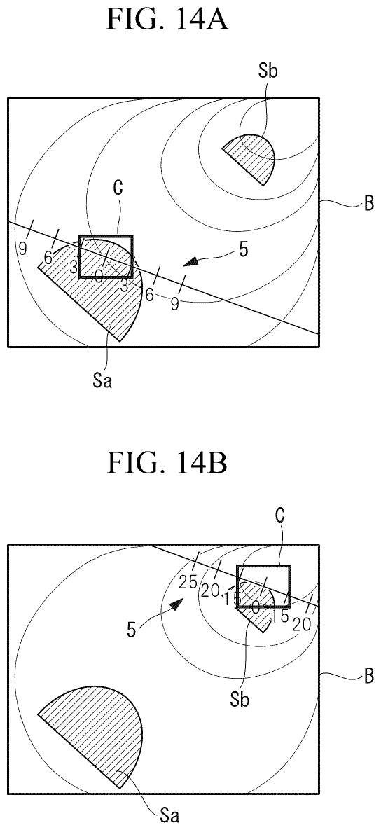

In FIGS. 12A to 13B, the linear scale extends parallel to the horizontal direction of the image B. Alternatively, as in FIGS. 14A and 14B, the linear scale may be inclined in the direction of distortion. In the case of FIGS. 14A and 14B, the actual distances from the origin are equal on the left side and the right side. Hence, a linear scale symmetrical with respect to the origin (that is, the interval between the scales and the numerical values 7 are the same on both sides of the origin) can be generated.

### Second Embodiment

Next, an image processing apparatus, an image processing method, and a storage medium according to a second embodiment of the present disclosure will be described.

This embodiment differs from the first embodiment in that the scale information is calculated using three-dimensional information of the object in addition to the object distance and the dimensional characteristics. In this embodiment, the configurations different from those in the first embodiment will be described, and the configurations common to those in the first embodiment will be denoted by the same reference signs, and the description thereof will be omitted.

Similarly to the first embodiment, the image processing apparatus 10 according to this embodiment is applied to the endoscope system 100 including the endoscope 20 and the display device 30, and includes a processor 101, a storage unit 2, a memory 3, and a user interface 4.

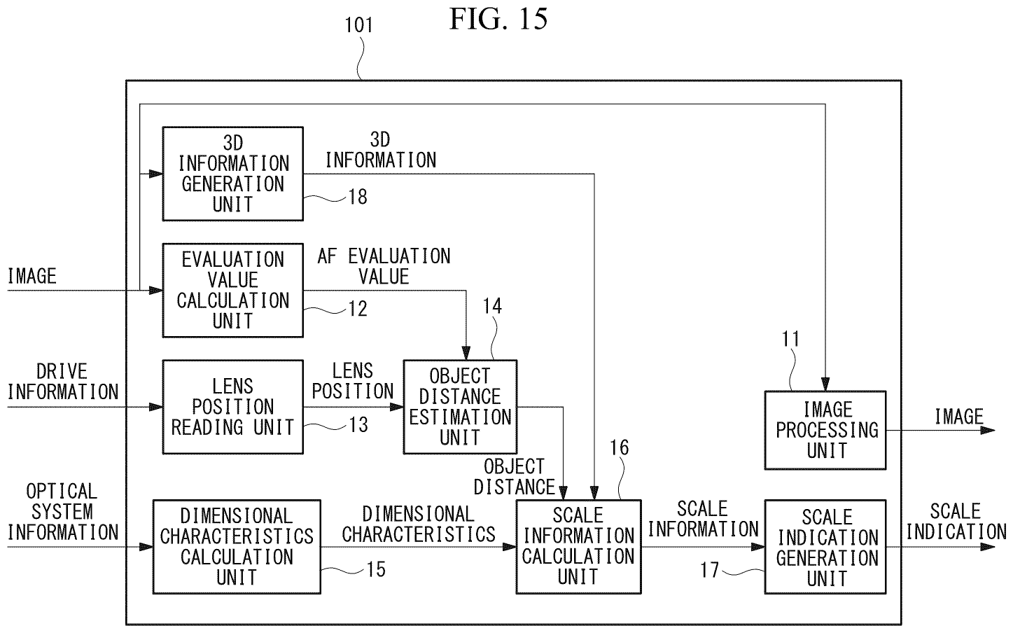

FIG. 15 is a functional block diagram of the processor 101 according to this embodiment.

The processor 101 includes, as functions, a three-dimensional (3D) information generation unit 18, in addition to the image processing unit 11, the evaluation value calculation unit 12, the lens position reading unit 13, the object distance estimation unit 14, the dimensional characteristics calculation unit 15, the scale information calculation unit 16, and the scale indication generation unit 17.

The 3D information generation unit 18 generates 3D information of an object S using multiple focused images viewed from different angles. The 3D information is a three-dimensional model of the object S and is generated using a known three-dimensional reconstruction technique. The positional relationship between the 3D information and the images used to generate the 3D information is known. The 3D information is defined by the relative dimensions of the object S and does not have information on the actual dimensions of the object S.

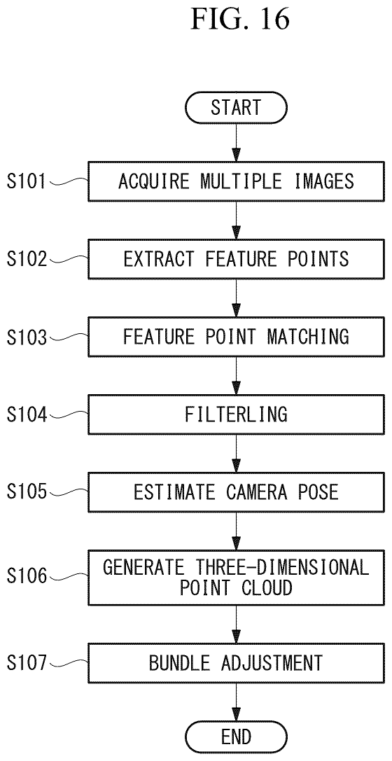

FIG. 16 shows an example method by which the 3D information generation unit 18 generates 3D information.

First, from images successively input to the image processing apparatus 10, multiple images for generating 3D information are acquired (step S101).

Next, feature points are extracted from each of the images (step S102). The feature points can be robust against changes, such as changes in magnification of the image, rotation, and illumination, and are extracted using a method such as scale-invariant feature transform (SIFT), Oriented FAST and Rotated BRIEF (ORB), or Accelerated KAZE (AKAZE).

Next, matching between the images is performed on the basis of the features of the feature points (step S103).

Next, from the matched feature points, erroneously matched feature points are removed by using a geometric constraint (step S104). A known method, such as a random sample consensus (RANSAC) algorithm, is used to remove the erroneously matched feature points.

Next, a change in the orientation of the camera is estimated on the basis of the change of the matched feature points (step S105). For example, when the intrinsic parameters of the camera are known, an essential matrix is estimated using five feature points, whereas when the intrinsic parameters are unknown, a fundamental matrix is estimated using eight or more feature points to estimate the orientation of the camera.

Next, the three-dimensional position of each of the matched feature points is estimated by means of triangulation based on the estimated essential matrix or fundamental matrix, by which a three-dimensional point cloud is generated (step S106).

Next, the orientation of the camera and the three-dimensional point cloud are adjusted (bundle adjustment is performed) by means of a nonlinear least squares method so that errors occurring when the point cloud is re-projected on the image are minimized (step S107).

The object distance estimation unit 14 determines the focus position of the AF lens 24 on the basis of the AF evaluation value and estimates the object distance on the basis of the focus position.

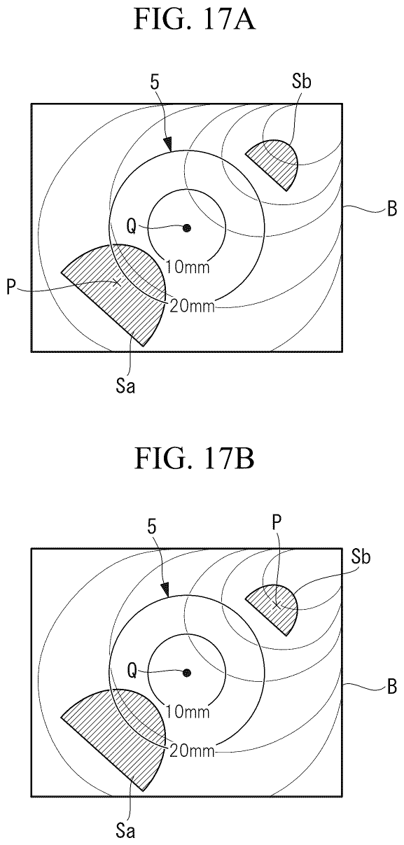

When the objects are distributed in the depth direction of the image, the center of the image is not necessarily focused, and another position may be focused. For example, as shown in FIG. 17A, the object Sa on the near side may be focused, or as shown in FIG. 17B, the object Sb on the far side may be focused. A point P indicates a focus position where the objects Sa and Sb are focused.

The object distance estimation unit 14 detects the focus position P in the image B on the basis of, for example, the AF evaluation values of the multiple ranging areas C in the image B, and estimates the object distance at the focus position P.

The scale information calculation unit 16 calculates scale information at a measurement point Q on the basis of the object distance at the focus position P, the dimensional characteristics, and the 3D information. The measurement point Q is a predetermined position in the image B. For example, as shown in FIGS. 17A and 17B, the measurement point Q is initially set at the center of the image B.

When the measurement point Q is the center of the image B, the scale information calculation unit 16 estimates the actual distance Z_Rd between the center of the image B and the focus position P from the object distance and the 3D information, using Equation (1) below.

Z_Rd=|Zf−Zo|×Z_Rf/Zf (1)
where Zo is the center position of the image in the 3D information, Zf is the focus position P in the 3D information, and Z_Rf is the object distance at the focus position P estimated by the object distance estimation unit 14.

Next, the scale information calculation unit 16 generates scale information on the basis of the distance Z_Rd and the dimensional characteristics.

The scale indication generation unit 17 generates, on the basis of the scale information, the scale indication 5 representing the actual dimensions at the measurement point Q, to be superimposed on the measurement point Q (for example, the center) in the image B.

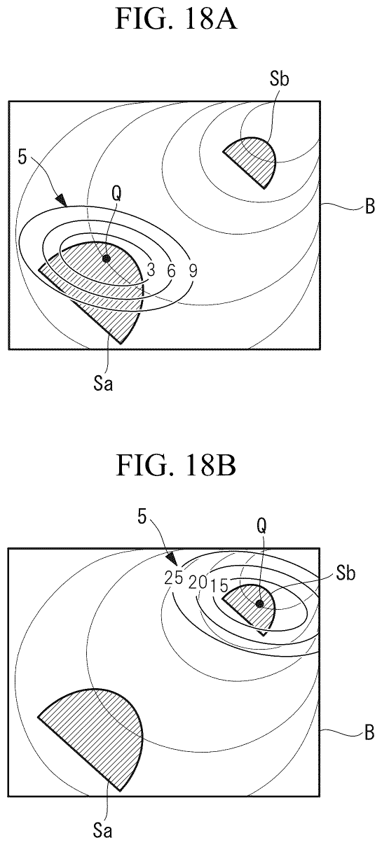

The scale information calculation unit 16 may set a position in the image B designated by the user as the measurement point Q. In that case, as shown in FIGS. 18A and 18B, the user operates the user interface 4 to designate a desired position in the image B. In FIGS. 18A and 18B, the object Sa on the near side and the object Sb on the far side are designated, respectively.

In this case, the scale information calculation unit 16 estimates the actual distance between the measurement point Q and the focus position P by using the position of the measurement point Q in the 3D information instead of the center position Zo in Equation (1). Next, the scale information calculation unit 16 generates scale information defining the relationship between the apparent distance from the measurement point Q and the actual distance from the measurement point Q on the basis of the estimated actual distance and the dimensional characteristics.

The scale indication generation unit 17 generates the scale indication 5 representing the actual dimensions at the measurement point Q on the basis of the scale information. The generated scale indication 5 is superimposed on the measurement point Q, for example, the object Sa or the object Sb.

The scale indication 5 is not necessarily concentric circles, and may be another scale indication 5 described in the first embodiment (see FIGS. 3A, 3B, and 10A to 14B). The size of the indication pattern 6 may be changed according to the object distance (see FIGS. 5A to 6B). Alternatively, the numerical values 7 may be changed according to the object distance (see FIGS. 7A to 8B), while the size of the indication pattern 6 is fixed. The scale indication 5 may be distorted according to the distortion of the image B (see FIGS. 10A to 11B, 14A, and 14B).

Next, an image processing method performed by the image processing apparatus 10 according to this embodiment will be described.

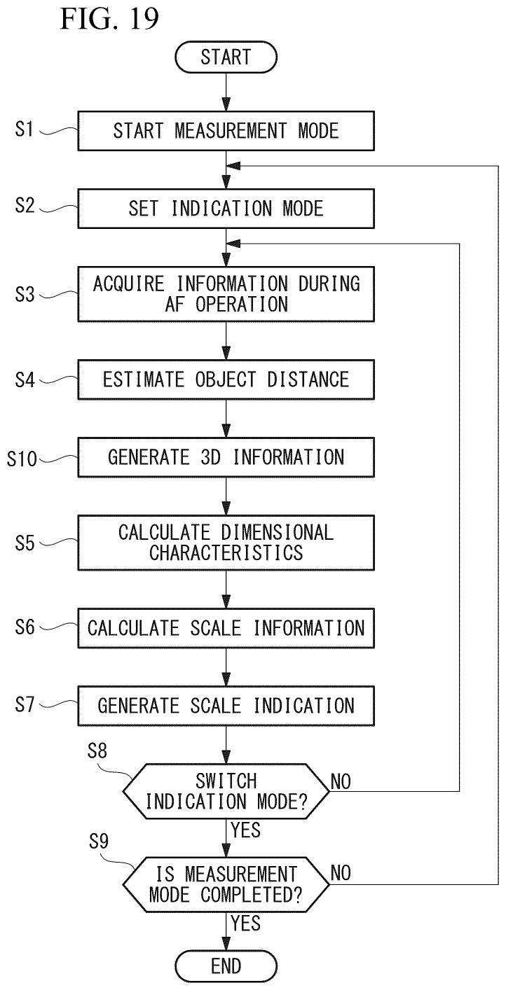

As shown in FIG. 19, the image processing method according to this embodiment includes step S10 of generating 3D information, in addition to steps S3 to S7.

After step S3, the object distance estimation unit 14 estimates the object distance at the focus position P in the image B (step S4). In parallel with this, the 3D information generation unit 18 generates 3D information (step S10).

Subsequently, after the dimensional characteristics are calculated (step S5), the scale information calculation unit 16 calculates the scale information at a predetermined measurement point Q, for example, the center, in the image B on the basis of the dimensional characteristics, the object distance, and the 3D information (step S6). Then, the scale indication generation unit 17 generates the scale indication 5 representing the actual dimensions of the object at the measurement point Q on the basis of the scale information (step S7). As a result, the image B in which the scale indication 5 is superimposed on the measurement point Q is displayed on the display device 30.

The user may situate the object to be measured at the measurement point Q, for example, the center, in the image B, and then start the measurement mode. In this way, the user can easily measure the actual dimensions of the object on the basis of the scale indication 5 at the measurement point Q in the image B.

When the user wishes to measure the dimensions of an object at another position, the user may designate a desired position in the image B by operating the user interface 4. In that case, the scale information calculation unit 16 calculates the scale information at the designated position (step S6), and the scale indication generation unit 17 generates the scale indication 5 at the designated position (step S7). As a result, the image B in which the scale indication 5 is superimposed at the designated position is displayed on the display device 30.

As described above, according to this embodiment, the scale information is calculated on the basis of the 3D information, in addition to the object distance and the optical characteristics. The 3D information includes information on the relative dimensions of the entire object in the image. Hence, it is possible to calculate information on the actual dimensions of the object at the measurement point Q located at a position different from the focus position P, for example, the actual distance from the focus position P to the measurement point Q, from the combination of the object distance and the 3D information. Then, the scale information for the measurement point Q at a desired position in the image B is calculated from the information on the actual dimensions and the dimensional characteristics, and the scale indication 5 is generated.

In this way, it is possible to generate the scale indication 5 at a desired position in the image B, regardless of the focus position P in the image B. In other words, when a user wishes to measure the dimensions of the object at a position different from the focus position P, the AF operation for focusing on the object is not necessary.

Furthermore, according to this embodiment, it is possible to simultaneously calculate the scale information at multiple positions in an image and simultaneously generate multiple scale indications 5. Hence, multiple measurement points Q may be set in the image B. For example, a user may simultaneously designate multiple positions, and multiple scale indications 5 may be simultaneously superimposed on the multiple designated positions. With this configuration, for example, the scale indication 5 in FIG. 18A and the scale indication 5 in FIG. 18B can be simultaneously superimposed on the image B.

Although the embodiments and modifications of the present disclosure have been described in detail with reference to the drawings, the specific configuration of the present disclosure is not limited to the above-described embodiments and modifications, and various design changes can be made without departing from the scope of the present disclosure. The components described in the above embodiments and modifications may be combined as appropriate.

For example, the scale indication 5 is not limited to the concentric circles or the linear scale described above, and may be of any other desired type representing the actual dimensions of the object S.

The above disclosure provides an advantage in that it is possible to measure the dimensions of an object without using a special endoscope.

1. An image processing apparatus for use with an endoscope, the image processing apparatus comprising:
a processor comprising hardware, the processor being configured to:
obtain an object distance;
calculate dimensional characteristics defining apparent dimensions of an object in an image acquired by the endoscope;
calculate scale information defining a relationship between the apparent dimensions and actual dimensions of the object based on the object distance and the dimensional characteristics; and
generate an indication pattern representing the actual dimensions based on the scale information.

2. The image processing apparatus according to claim 1, wherein the processor is configured to change a size of the indication pattern in accordance with the object distance.

3. The image processing apparatus according to claim 1, wherein the processor is configured to generate the indication pattern having a fixed size and change a numerical value representing the actual dimensions attached to the indication pattern in accordance with the object distance.

4. The image processing apparatus according to claim 1, wherein the indication pattern comprises a plurality of concentric circles having radii different from each other.

5. The image processing apparatus according to claim 1, wherein the indication pattern is a linear scale.

6. An image processing apparatus for use with an endoscope, the image processing apparatus comprising:
a processor comprising hardware, the processor being configured to:
calculate an object distance based on a focus position of a focus lens, the focus position being a position when an object is focused;
calculate dimensional characteristics defining apparent dimensions of the object based on optical system information of an imaging optical system of the endoscope in a state in which the focus lens is located at the focus position;
generate three-dimensional information of the object using an image acquired by the endoscope;
calculate scale information defining a relationship between the apparent dimensions and actual dimensions of the object based on the object distance, the dimensional characteristics, and the three-dimensional information; and
generate an indication pattern representing the actual dimensions based on the scale information, the indication pattern configured to be superimposed on the image.

7. The image processing apparatus according to claim 6, wherein the processor is further configured to:
calculate the scale information defining the relationship between the apparent dimensions and the actual dimensions of the object at a measurement point in the image; and
generate the indication pattern representing the actual dimensions at the measurement point based on the scale information, wherein the indication pattern configured to be superimposed on the measurement point.

8. The image processing apparatus according to claim 7, wherein the processor is configured to set a position in the image designated by a user as the measurement point.

9. The image processing apparatus according to claim 6, wherein the processor is configured to change a size of the indication pattern in accordance with the object distance.

10. The image processing apparatus according to claim 6, wherein the processor is configured to generate the indication pattern having a fixed size and change a numerical value representing the actual dimensions attached to the indication pattern in accordance with the object distance.

11. The image processing apparatus according to claim 6, wherein the indication pattern comprises a plurality of concentric circles having radii different from each other.

12. The image processing apparatus according to claim 6, wherein the indication pattern is a linear scale.

13. An image processing method for use with an endoscope, the image processing method comprising:
calculating an object distance based on a focus position of a focus lens, the focus position being a position when an object is focused;
calculating dimensional characteristics defining apparent dimensions of the object based on optical system information of an imaging optical system of the endoscope in a state in which the focus lens is located at the focus position;
calculating scale information defining a relationship between the apparent dimensions and actual dimensions of the object based on the object distance and the dimensional characteristics; and
generating an indication pattern representing the actual dimensions based on the scale information, the indication pattern configured to be superimposed on an image acquired by the endoscope.

14. An image processing method for use with an endoscope, the image processing method comprising:
calculating an object distance based on a focus position of a focus lens, the focus position being a position when an object is focused;
calculating dimensional characteristics defining apparent dimensions of the object based on optical system information of an imaging optical system of the endoscope in a state in which the focus lens is located at the focus position;
generating three-dimensional information of the object using an image acquired by the endoscope;
calculating scale information defining a relationship between the apparent dimensions and actual dimensions of the object based on the object distance, the dimensional characteristics, and the three-dimensional information; and
generating an indication pattern representing the actual dimensions based on the scale information, the indication pattern configured to be superimposed on the image.

15. A non-transitory computer-readable storage medium storing instructions that cause a computer to perform:
calculating an object distance based on a focus position of a focus lens, the focus position being a position when an object is focused;
calculating dimensional characteristics defining apparent dimensions of the object based on optical system information of an imaging optical system of an endoscope in a state in which the focus lens is located at the focus position;
calculating scale information defining a relationship between the apparent dimensions and actual dimensions of the object based on the object distance and the dimensional characteristics; and
generating an indication pattern representing the actual dimensions based on the scale information, wherein the indication pattern configured to be superimposed on an image acquired by the endoscope.

16. A non-transitory computer-readable storage medium storing instructions that cause a computer to perform:
calculating an object distance based on a focus position of a focus lens, the focus position being a position when an object is focused;
calculating dimensional characteristics defining apparent dimensions of the object based on optical system information of an imaging optical system of an endoscope in a state in which the focus lens is located at the focus position;
generating three-dimensional information of the object using an image acquired by the endoscope;
calculating scale information defining a relationship between the apparent dimensions and actual dimensions of the object based on the object distance, the dimensional characteristics, and the three-dimensional information; and
generating an indication pattern representing the actual dimensions based on the scale information, wherein the indication pattern configured to be superimposed on the image.

17. The image processing apparatus according to claim 1, wherein the obtaining the object distance comprises:
calculating the object distance based on a focus position of a focus lens, the focus position being a position when the object is focused.

18. The image processing apparatus according to claim 1, wherein the object distance is a distance between an objective lens and the object.

19. The image processing apparatus according to claim 1, wherein the calculating the dimensional characteristics comprises:
calculating the dimensional characteristics defining apparent dimensions of the object based on optical system information of an imaging optical system of the endoscope in a state in which a focus lens is located at a focus position.

20. The image processing apparatus according to claim 1, wherein the processor is configured to:
superimpose the indication pattern on the image.
